(19) United States
(12) Patent Application Publication
(10) Pub. No.: US 2006/0217664 A1
(43) Pub. Date: Sep. 28, 2006

(54) Telescoping vascular dilator

(76) Inventors: Brack G. Hattler, Pittsburgh, PA (US); Vincent J. Testa, Cranberry Township, PA (US)

Correspondence Address: FULWIDER PATTON, 6060 CENTER DRIVE, 10TH FLOOR, LOS ANGELES, CA, 90045, US

(21) Appl. No.: 11/251,322

(22) Filed: Oct. 14, 2005

Related U.S. Application Data
(60) Provisional application No. 60/628,309, filed on Nov. 15, 2004.

(51) Int. Cl. A61M 5/178

(52) U.S. Cl. 604/164.1; 604/164.05

(57) ABSTRACT

A telescoping dilator assembly is configured for percutaneous insertion of large diameter intravascular devices into a blood vessel while avoiding kinking of a guidewire and minimizing blood loss. The assembly includes a first smaller diameter dilator with an inner lumen that fits snugly over a guidewire. The smaller dilator may be tapered on both ends. A second larger diameter dilator is configured to slide over the first smaller dilator. A tear away sheath is configured to slide over the dilators and into a blood vessel. A tear away sheath plug is configured to form a seal at a proximal end of the sheath. When the dilators are removed from the blood vessel, an intravascular device may be passed through the sheath into the blood vessel.

### CROSS-REFERENCES TO RELATED APPLICATION

This application claims priority to U.S. Provisional Patent Application Ser. No. 60/628,309 filed Nov. 15, 2004 entitled HEMOSTASIS DILATOR, the entire contents of which are incorporated herein by reference.

### FEDERALLY SPONSORED RESEARCH

The U.S. Government has a paid-up license in this invention and the right in limited circumstances to require the patent owner to license others on reasonable terms as provided for by the terms of Grant Nos. DAMD 17-03-1-0512 awarded by the Department of the Army.

### BACKGROUND OF THE INVENTION

This invention relates to the field of dilators used for insertion of medical instruments and devices into the body. More specifically, this invention relates to a system of vascular dilators, wherein one dilator may be telescopically inserted over another smaller diameter dilator, thereby slowly enlarging an entry point into the body. The invention is useful for establishing intravascular access for insertion of large diameter intravascular devices into the body through a sheath, while avoiding kinking of a guidewire. The invention further includes a tear away sheath and a tear away sheath plug.

Several medical devices require percutaneous insertion into the body. Larger diameter intravascular devices present a special challenge for minimally invasive insertion into a blood vessel without a surgical “cut down” to the vessel. Percutaneous insertion of a catheter into a blood vessel has typically been accomplished by first passing a hypodermic needle into the blood vessel and then inserting a guidewire through the interior of the hypodermic needle. The percutaneous entry point is then expanded by passing one dilator over the guidewire until the percutaneous entry point enlarges sufficiently to permit the passage of a sheath into the blood vessel, and finally the passage of a medical device through the sheath.

Problems with the prior art dilators and sheaths occur when a very large diameter sheath is required to insert large diameter medical devices into a blood vessel. For example, respiratory assist catheters known in the art may be inserted via a large peripheral vein into central veins, for example, the vena cava. These types of devices are preferably percutaneously inserted into the peripheral vein, for example, the femoral vein, through the interior of a previously inserted sheath, and then advanced centrally into the vena cava. Because respiratory assist catheters may have a fixed large diameter, the interior and exterior diameters of the corresponding sheath must likewise be quite large.

Dilators described in the prior art have problems when used for insertion of such a large sheath and large diameter medical device into a venous structure. Dilators have a distal end portion that is typically inserted into the patient, and a proximal end portion that remains outside of a patient. The dilators that are described in the known prior art are tapered at the distal end with a larger diameter proximal end. The taper may be gradual along the entire length of the dilator, but, more commonly, the taper is at the distal segment and the remainder of the dilator has a consistent tubular dimension. In either configuration, the smaller diameter distal end of the dilator is first typically inserted over a guidewire, and the dilator then advanced over the guidewire into the vascular structure. The proximal segment of the dilator remains external to the patient's body. Since the wall of the dilator is a constant thickness, an inner channel of the dilator increases in diameter from distal to proximal. Therefore, the space around the guidewire, that is the distance between the dilator interior wall and the guidewire, increases from distal to proximal. This space may become quite large with larger diameter dilators, such as those used with large diameter sheaths and large diameter intravascular devices. One problem is that the guidewire may kink, bend, knot, or otherwise be damaged within the large inner channel within the dilator.

Splittable or tear apart sheaths, and cannulas, are also known in the prior art. The splittable feature of a sheath may be useful when the sheath cannot be slipped off the end of an introducer. The splittable sheath may be circumferentially disposed about the dilator which, in turn, may be circumferentially disposed about the wire guide. The distal end of the dilator is tapered for enlarging a puncture site to permit a vein to accommodate the splittable sheath. In one known dilator, where the diameter of the splittable sheath is of a sufficiently large size, the single dilator that is used has a second proximal tapered portion. However, as described, both of these tapers are included on the same dilator. The tubular portion of the splittable sheath has substantially uniform wall thickness and inner diameter except at its distal end where there is a slight taper to create an appropriately snug fit with the dilator. The taper also facilitates the enlarging of the puncture site to allow insertion of the sheath. However, the inner channel of the single dilator with a double taper described in the known prior art, if made in a large diameter dilator, would still be much larger than the enclosed guidewire at the proximal end. The guidewire may still be subject to kinking. Therefore, the described devices do not solve the problem of how to safely use a dilator with a medical device requiring a very large sheath. The known prior art suggestion of putting a second taper on the outside of the one dilator for use with larger sheaths does not recognize or solve the problem of guidewire kinking. More specifically, the known prior art does not address how to prevent guidewire kinking within the large free space of the inner channel of a large diameter dilator. Prior known devices do not disclose use of a second dilator.

Also previously disclosed is a catheter dilator sheath assembly. The known device has an interlocking superimposed sheath and dilator that provides a two step gentle enlargement of a puncture site and a stable axial relationship between the sheath and the dilator. However, the referenced device also does not recognize or solve the problem of guidewire kinking within the large inner channel of a large dilator.

Also known in the art is a cannula and obturator combination. The disadvantage of this device is that the tip of the obturator has a rounded blunt end and an open surgical incision must be performed for access into a venous channel. The cannula and obturator cannot be inserted percutaneously through a needle puncture. The cannula is hollow, tubular, and tapered. The obturator fits within the cannula and has a distal tip. The cannula and obturator are inserted through a single incision. The described device however is not designed to be percutaneously inserted into the body over a guidewire.

Hence, those skilled in the art have recognized a need for a device and method that allows large intravascular medical devices to be percutaneously passed into a blood vessel through a sheath, with minimal skill and without the need for a surgical incision. Those skilled in the art have also recognized a need for a device that allows for a large sheath to be inserted percutaneously over a guidewire, while avoiding kinking of the guidewire within a dilator. The present invention fulfills these needs and others.

### SUMMARY OF THE INVENTION

Briefly and in general terms, the present invention provides a dilator assembly that allows large intravascular devices to be percutaneously passed into a blood vessel or other body cavity without the need for a surgical incision and with minimal blood loss during dilation of the blood vessel. The present invention further provides a dilator and sheath combination that allows large intravascular devices to be percutaneously passed into a blood vessel or other body cavity through the sheath without the need for a surgical incision. One aspect of the present invention is a minimally invasive procedure for a patient, and less surgical skill required by a clinician for insertion of large intravascular medical devices into a blood vessel. The present invention also provides a method of inserting a large sheath into a blood vessel percutaneously over a guidewire while avoiding kinking of the guidewire. The method of the present invention further includes insertion of at least one embodiment of a respiratory assist catheter through the sheath and into the vena cava of a patient.

The present invention provides a telescoping dilator assembly and sheath combination that allows large intravascular medical devices to be passed into a blood vessel or other body cavity through the sheath, by percutaneous puncture of the vessel. The advantage is that percutaneous puncture may be performed in a radiology suite or an ICU setting. The patient does not need to be moved to an operating room. A surgical cut down is not required for insertion of large intravascular medical devices when using the present invention.

The present invention also provides an introducer assembly for the insertion of a large diameter sheath, catheter, cannula, or other device into a blood vessel, while avoiding damage to or kinking of a guidewire. Gradual enlargement of the percutaneous entry site into a blood vessel may be accomplished by passing sequentially larger dilators over a guidewire, until a large diameter sheath can be passed into the blood vessel over the last dilator. The invention further provides a tear away sheath plug, that may be useful when inserting a respiratory assist catheter into a blood vessel through a large diameter sheath.

The present invention includes an improved dilator and sheath combination, having at least two dilators and a sheath, useful for introduction of very large sheaths into the body. The dilator and sheath combination may be used for inserting larger medical devices into a body cavity, such as a blood vessel. The invention includes at least one smaller diameter dilator and at least one larger diameter dilator. The smaller diameter dilator may be, for example, eleven French and the larger diameter dilator may be, for example, thirty-four French. The smaller diameter dilator may be passed over a guidewire that has been percutaneously inserted into a large peripheral vein. For example, the guidewire may be inserted from the femoral vein, into the vena cava.

The invention further includes a first smaller diameter dilator that may be tapered on both ends, either end of which may be interchangeably inserted into the vein. The smaller diameter dilator is sufficiently long, for example, about thirty-six inches (ninety-two centimeters) long in at least one embodiment, to allow approximately eighteen inches (forty-six cm) of the smaller diameter dilator to extend outside of the percutaneous entry point. This enables a second larger diameter dilator, for example, about fourteen inches (thirty-six cm) long in at least one embodiment, to be passed over both the smaller diameter dilator and the indwelling guidewire, while retaining proximal control over the smaller diameter dilator. A sheath may be disposed around the second larger diameter dilator. The sheath may be slidingly disposed around the second larger diameter dilator. Approximately two to three inches (five to eight cm) of distal tapered end on the larger diameter dilator may be exposed beyond the sheath. The remaining proximal tubular part of the larger diameter dilator may be surrounded by the sheath during insertion.

The larger diameter dilator may be in at least one embodiment preassembled and detachably locked by a hub to the sheath. When the sheath is in place, for example, in the femoral vein or in the iliac vein, the two dilators and guidewire are removed from the interior of the sheath. The large medical device, for example, a respiratory assist catheter, may then be inserted through the sheath and advanced into the vena cava. The sheath may be designed to be longitudinally split into at least two parts and configured to tear away from the respiratory assist catheter, leaving only the respiratory assist catheter in the patient. The introducer assembly of the present invention may include a sheath plug having a substantially cylindrical body, a substantially unobstructed longitudinal passageway through the cylindrical body and an enlarged proximal segment. The sheath plug may be configured with two tabs disposed on the enlarged proximal segment so as to tear the sheath plug along a longitudinal axis.

The present invention may further include any number of additional dilators. For example, at least one additional dilator may be passed over the first smaller diameter dilator before passing the second larger diameter dilator. In yet another embodiment, at least one additional dilator may be passed over the second larger diameter dilator.

The present invention provides a dilator assembly, including a first dilator configured with a first proximal portion having a first proximal aperture, a first tapered distal portion having a first distal aperture, and a first tubular segment having a substantially uniform first outer diameter and a first inner lumen between the first proximal portion and the first distal portion. The dilator assembly further includes a second dilator configured with a second proximal portion having a second proximal aperture, a second tapered distal portion having a second distal aperture, and a second tubular segment between the second proximal portion and the second distal portion, the second tubular segment having a substantially uniform second outer diameter and a second inner lumen, wherein the diameter of the second inner lumen is substantially the same as the outer diameter of the first tubular segment of the first dilator.

The invention further provides an introducer assembly, including a first dilator configured with a first proximal portion having a first proximal aperture, a first tapered distal portion having a first distal aperture, and a first tubular segment having a substantially uniform first outer diameter and a first inner lumen between the first proximal portion and the first distal portion. The introducer assembly further includes a second dilator configured with a second proximal portion having a second proximal aperture, a second tapered distal portion having a second distal aperture, and a second tubular segment between the second proximal portion and the second distal portion, the second tubular segment having a substantially uniform second outer diameter and a second inner lumen, wherein the diameter of the second inner lumen is substantially the same as the outer diameter of the first tubular segment of the first dilator. The introducer assembly also includes a sheath configured with a third inner diameter substantially the same as the second outer diameter of the second dilator.

The invention further provides a method of dilating an opening into a blood vessel, including inserting a guidewire into a blood vessel, inserting a first dilator into blood vessel over the guidewire, and inserting a second dilator into blood vessel over the first dilator.

The invention yet further provides a method of inserting a medical device into a blood vessel, comprising inserting a guidewire into a blood vessel, sliding a first dilator over the guidewire, sliding a second dilator over the first dilator, inserting a sheath over the second dilator, removing the first dilator and the second dilator from the blood vessel, and inserting a medical device into the blood vessel through a proximal opening of the sheath.

The invention yet further includes a dilator kit, including a guidewire and a first dilator configured with a first proximal portion having a first proximal aperture, a first tapered distal portion having a first distal aperture, and a first tubular segment having a substantially uniform first outer diameter and a first inner lumen between the first proximal portion and the first distal portion. The dilator kit further includes a second dilator configured with a second proximal portion having a second proximal aperture, a second tapered distal portion having a second distal aperture and a second tubular segment between the second proximal portion and the second distal portion, the second tubular segment having a substantially uniform second outer diameter and a second inner lumen, wherein the diameter of the second inner lumen is substantially the same as the outer diameter of the first tubular segment of the first dilator.

The invention further includes a method of inserting a medical device into a patient's blood vessel including inserting a guidewire into a blood vessel, sliding a first dilator into the blood vessel over the guidewire, sliding one or more additional larger diameter dilators over the first dilator into the blood vessel, and passing a sheath over the second dilator into the blood vessel. The method further includes removing the guidewire, first dilator, and additional dilator(s) from the sheath, and passing the medical device through the sheath into the blood vessel. The method may further include applying a slide clamp to prevent backflow of blood through the sheath and releasing the slide clamp to allow passage of the medical device through the sheath. The method may further include inserting a sheath plug into the sheath to prevent bleeding around the medical device, between the proximal segment of the medical device and the inner wall of the sheath.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a plan side view of the medical dilator assembly of the present invention.

FIG. 2 is a partial cross-sectional view of the medical dilator assembly of FIG. 1.

FIG. 3A is a perspective view of at least one embodiment of a sheath plug.

FIG. 3B is a side plan view of the sheath plug shown in FIG. 3A.

FIG. 3C is a top plan view of the sheath plug shown in FIG. 3A.

FIG. 3D illustrates the sheath plug shown in FIG. 3A after it is axially split.

FIG. 3E illustrates a large diameter medical device passing through a hollow central passageway of the sheath plug shown in FIG. 34A.

FIG. 4 is a perspective view of a slide clamp of the present invention.

FIG. 5 illustrates a guidewire being inserted through a hypodermic needle into a vein of a patient.

FIG. 6 depicts an assembly of a first smaller diameter dilator, a second larger diameter dilator, and a sheath being passed over a guidewire into a peripheral vein.

FIG. 7 depicts a dilator assembly of the present invention being advanced over a guidewire, wherein a first smaller diameter dilator and a distal portion of a second larger diameter dilator have entered into a peripheral vein.

FIG. 8 depicts a dilator assembly of the present invention being advanced over a guidewire, wherein a distal portion of the sheath has entered into a peripheral vein.

FIG. 9 depicts a sheath in a peripheral vein over a guidewire disposed in a peripheral vein.

FIG. 10 depicts a distal portion of a respiratory assist catheter being inserted into a sheath disposed in a peripheral vein.

FIG. 11 depicts a respiratory assist catheter having been advanced into a vein and a sheath plug inserted into a proximal end of a sheath disposed in the vein.

FIG. 12 shows a split sheath plug, and a split sheath around a respiratory assist catheter disposed in a blood vessel.

FIG. 13 is a kit including a guidewire, a smaller diameter dilator, a larger diameter dilator, a sheath, and a sheath plug.

FIG. 14 is a perspective view of a guidewire passing through a dilator having a hemostasis seal.

### DETAILED DESCRIPTION OF THE INVENTION

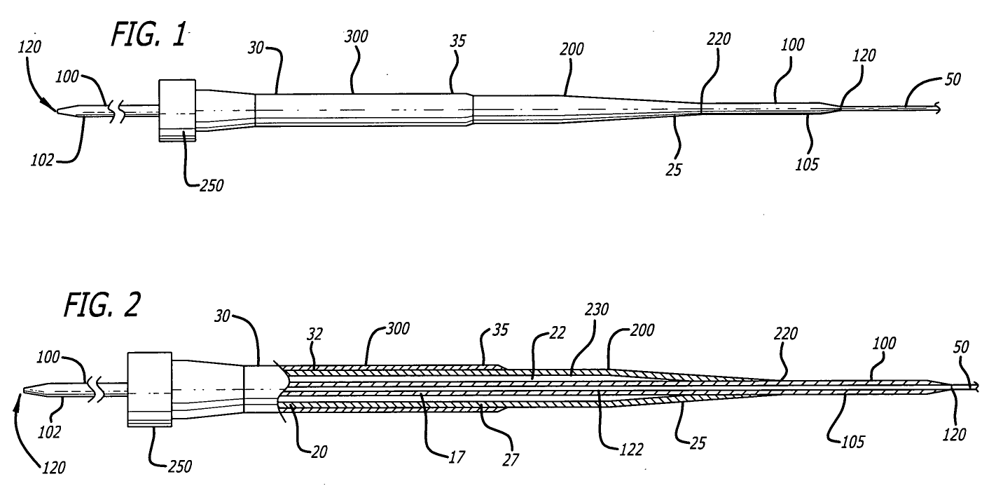

Referring to FIGS. 1 and 2, the present invention includes at least one larger diameter dilator 200 that may be circumferentially and telescopically passed over at least one smaller diameter dilator 100. The smaller diameter dilator 100 may be circumferentially passed over a guidewire 50 that has been percutaneously inserted into a blood vessel. Because an inner channel 122 of the smaller diameter dilator fits snugly around the guidewire 50, once the smaller diameter dilator 100 has been passed over the guidewire 50, the guidewire 50 may be prevented from kinking. The larger diameter dilator 200 can then safely be passed over the smaller diameter dilator 100. The larger diameter dilator 200 may be further detachably connected with a sheath 300 by a fitting 250 that detachably locks the larger diameter dilator 200-and sheath 300 together in position for insertion into the blood vessel. The larger diameter dilator 200 is capable of being unlocked from the sheath 300 after insertion into a patient. The smaller diameter dilator 100, the larger diameter dilator 200, and the guidewire 50 can then be withdrawn from the interior of the sheath 300, leaving the sheath 300 remaining in the blood vessel. A variety of large diameter intravascular devices can then be inserted into the patient through the sheath 300. In at least one embodiment of the invention, the larger diameter dilator 200 may be part of an assembly including a larger diameter dilator 200 and a sheath 300. The dilators may be made from biocompatible materials, for example, a medical grade plastic or silicone material that may be covered with a non-stick surface, for example, polytetrafluoroethylene (PTFE). It should be understood that the description of two dilators is by way of example only, and in other embodiments, at least one additional dilator may be passed over the second larger diameter dilator 200. In other embodiments, the invention may further include any number of additional dilators with incrementally larger diameters. For example, at least one additional dilator with a larger diameter than the first smaller diameter dilator may be passed over the first smaller diameter dilator, before passing the second larger diameter dilator. In yet another embodiment, at least one additional dilator may be passed over the second larger diameter dilator.

An advantage of the present invention is a reduction in blood loss associated with a procedure of dilating an opening in a blood vessel when using the dilators of the present invention as compared to serial dilators known in the prior art. The serial dilators known in the prior art must each be withdrawn from the blood vessel before another dilator can be inserted into the blood vessel. Withdrawing the serial dilator results in bleeding from the opening in the blood vessel created by the dilator. Each time a serial dilator is withdrawn, blood loss occurs from the patient, around the guidewire 50, because the opening in the blood vessel from the dilator is not sealed until another serial dilator is passed. The opening in the blood vessel may be become quite large when using a large dilator, for example, a thirty-four French dilator. In contrast, the larger diameter dilator 200 of the present invention may be telescopically passed over the at least one smaller diameter dilator 100. The smaller diameter dilator 100 need not be withdrawn from the blood vessel before passing the larger diameter dilator 200. Therefore, the opening in the blood vessel is always sealed and less blood loss results from the procedure.

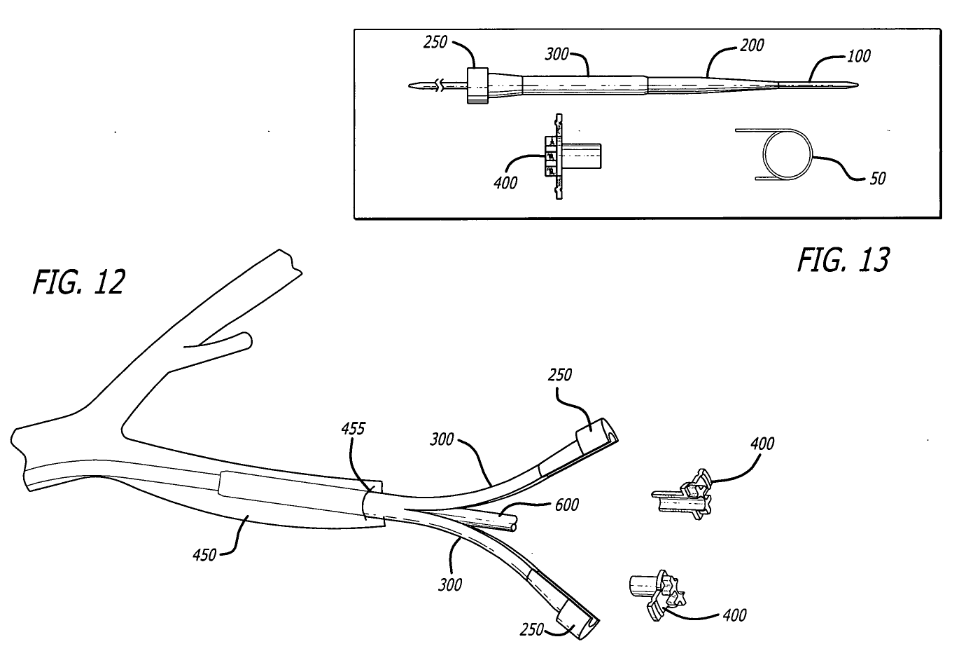

The invention may be useful for insertion of large diameter intravascular devices. For example, the invention may be useful for insertion of a respiratory assist catheter 600, as partially shown in FIGS. 11-13. At least one example of a type of respiratory assist catheter suitable for use with the present invention is described in detail in U.S. Pat. Nos. 5,376,069 and 5,501,663 which are herein incorporated by reference in their entirety. The inclusion of the respiratory assist catheter 600 is by way of example only and is not intended to be limiting to the invention. It will be recognized by those skilled in the art that the invention will also be applicable to dilation of other anatomic areas of the body and the percutaneous or open insertion of other large intravascular devices. It will also be recognized that the invention is not limited to intravascular use, and may be applicable to insertion of any large diameter medical device that requires insertion into a body cavity through a minimally invasive or percutaneous opening.

The first smaller diameter dilator 100 may be longer than most medical dilators currently on the market. The smaller diameter dilator 100 in at least one embodiment is about thirty-six inches (ninety-two cm) long. This permits the smaller diameter dilator 100 to be controlled by the clinician during insertion of the larger diameter dilator 200 over the smaller diameter dilator 100. In one embodiment, the smaller diameter dilator 100 has a diameter of size eleven French.

Still referring to FIGS. 1 and 2, the first smaller diameter dilator 100 has a proximal end 102 and a distal end 105, and may have a substantially uniform wall thickness. The proximal end 102 and the distal end 105 each have an aperture 120 that opens into an inner channel 122 within the smaller diameter dilator 100. The apertures 120 and inner channel 122 are of appropriate diameter to allow the passage of a variety of guidewires 50. In at least one embodiment, a 0.038 inch (one mm) super-stiff guidewire may be preferred. The inner channel 122 may fit securely and relatively snuggly around the guidewire 50. The fit is snug enough to assure that the guidewire cannot kink within the inner channel 122 of the smaller diameter dilator 100, while still permitting the guidewire to easily slide axially within the inner channel 122. The inner channel 122 is small enough in diameter to not permit kinking of the guidewire 50.

Still referring to FIGS. 1 and 2, the first smaller diameter dilator 100, in at least one embodiment, may be tapered over both the proximal end 102 and the distal end 105. In at least one embodiment, approximately two to three inches (five to eight cm) of both the proximal end 102 and the distal end 105 are tapered. Alternatively, only the distal end 105 or only the proximal end 102 may be tapered. The portion of the smaller diameter dilator 100 between the proximal end 102 and the distal end 105 includes a uniform tubular segment 17. Although the smaller diameter dilator 100 need only be tapered on one end, a bilateral taper may be advantageous. The bilateral taper makes it efficient for the clinician to insert either end of the smaller diameter dilator 100 over the guidewire and into the patient without having to first examine the ends of the smaller diameter catheter. This is because the ends of the smaller diameter catheter are symmetrical and interchangeable. The opposite tapered end, that is the end that is not inserted into the patient, then acts as a guide for the initial passage of the second larger diameter dilator 200 over the first smaller diameter dilator 100. In at least one embodiment, the smaller diameter dilator is made of high density polyethylene (HDPE) that has been doped with approximately eight percent to fifteen percent barium sulfate (BaSO₄) that enables the device to be visible under x-ray or fluoroscopy.

Referring more specifically now to FIG. 2, the second larger diameter dilator 200 has a proximal end 20 and a distal end 25 with an inner channel 22 therebetween. The distal end 25 has an aperture 220 through which the smaller diameter dilator 100 is capable of passing into and through the inner channel 22. The proximal end 20 also has an aperture (not shown) that is, in at least one embodiment, larger than the aperture 220 in the distal end 25. The inner channel 22 at the proximal end 20 of the second larger diameter dilator 200 may have a diameter that is at least twice as great as the outer diameter of the tubular segment 17 of the first smaller diameter dilator 100. In one embodiment, the large diameter dilator 200 is approximately fourteen inches (thirty-six cm) long having a diameter of size thirty-four French. The distal end 25 of the larger diameter dilator 200 may be tapered, for example, over approximately two to three inches (five to eight cm). The proximal end 20 of the larger diameter dilator includes a tubular segment 27 that in at least one embodiment has a substantially uniform untapered diameter. The aperture 220 in the distal end 25 may be smaller than the aperture (not shown) in the proximal end 20. Any longitudinal location on the second dilator 200 will have a greater inner diameter than the largest outer diameter of the first dilator 100, thereby permitting the second dilator to be slidably disposed on the first dilator. The wall thickness of the larger diameter dilator 200 may be uniform or may vary over the length of the dilator to add stiffness to regions of the larger diameter dilator 200. In at least one embodiment, the second larger diameter dilator 200 is made of low density polyethylene (LDPE) that has also been doped with approximately eight percent to fifteen percent barium sulfate (BaSO₄).

The aperture 220 at the distal end 25 of the second larger diameter dilator 200 preferably fits snugly around the uniform tubular segment 17 of the first smaller diameter dilator 100. The snug fit allows easier insertion of the larger diameter dilator 200 into the blood vessel and prevents the reflux of blood between the smaller diameter dilator 100 and the larger diameter dilator 200. The smaller diameter dilator 100 is capable of sliding longitudinally through the inner channel 22 of the larger diameter dilator 200. The larger diameter dilator 200 is configured to telescopically or circumferentially slide over the smaller diameter dilator 100. In one embodiment, the larger diameter dilator 200 is tapered and has a uniform wall thickness. The inner channel 22 of the larger diameter dilator 200 increases in size from distal to proximal. When the larger diameter dilator 200 is positioned over the smaller diameter dilator 100, the space around the smaller diameter dilator 100 increases from distal to proximal. The guidewire 50 is prevented from kinking because the guidewire is surrounded and restricted within the smaller diameter dilator 100.

Still referring to FIGS. 1 and 2, the invention may further include an additional device that is configured to be slidably disposed over the larger diameter dilator 200, for example, a sheath 300. The term sheath as used herein includes any device that may be introduced over the dilators, such as a catheter, an additional dilator, or a cannula. The sheath 300 in one preferred embodiment may have a thirty-four French inner diameter and a thirty-eight French outer diameter. The sheath 300 has a proximal end 30 and a distal end 35. The sheath 300 may be of uniform diameter and wall thickness, or may be tapered. In one embodiment, the sheath 300 is configured to be split down the longitudinal axis. In at least one other embodiment, the sheath 300 may be configured to not be split down the longitudinal axis. The distal end of the sheath 300 may be slightly tapered to aid in insertion through the percutaneous entry point and into the blood vessel. An inner channel 32 of the sheath 300 may be configured to fit snuggly over the proximal segment 20 of the larger diameter dilator 200. The sheath 300 is designed to be slidably disposed on the larger diameter dilator 200. The proximal end 30 of the sheath 300 and the proximal end 20 of the larger diameter dilator 200 may be detachably locked together by a fitting 250 prior to insertion into the blood vessel. Various types of locking mechanisms, all of which are well known in the art, may be utilized for the fitting 250, which detachably locks the sheath 300 to the larger diameter dilator 200. After the sheath 300 and larger diameter dilator 200 are unlocked, the sheath 300 can slide over the larger diameter dilator 200, thereby enabling separation and removal of the larger diameter dilator 200 from the sheath 300. The sheath may be made from various materials, including, but not limited to, polytetrafluoroethylene (PTFE), polyethylenes, polyvinylchlorides (PVC), polyurethanes, or silicones.

The second larger diameter dilator 200 and the sheath 300 may be pre-assembled in the connected, locked, and axially aligned position. The fitting 250 may detachably lock the most proximal 20 end of the larger diameter dilator 200 with the most proximal 30 end of the sheath 300. The fitting 250 provides for a locked larger diameter dilator 200 and sheath 300 assembly that is configured for insertion into a patient's blood vessel. After insertion of the sheath 300 into the blood vessel, the larger diameter dilator 200 may be unlocked from the sheath 300. The larger diameter dilator 200, smaller diameter dilator 100, and guidewire 50 may then be removed through the sheath 300 from the blood vessel. Various intravascular devices can then be passed into the patient's blood vessel through the inner channel or lumen 32 of the sheath 300.

The sheath 300 of the present invention may be configured as a tear-apart sheath. A tear apart sheath is a sheath that may be longitudinally split into at least two parts. An advantage of a tear-apart sheath is that the sheath 300 can be completely removed from around a medical device after the insertion of the medical device through the sheath 300. Tabs or knobs (not shown) may be disposed on the proximal 30 end of the sheath 300 to help initiate the splitting of the sheath 300. As pressure is applied to the tabs or knobs, the sheath 300 will split from the proximal end 30 to the distal end 35. Such tabs and knobs are known in the art and need not be described in more detail. Furthermore, the sheath may be axially scored to assist the splitting of the sheath.

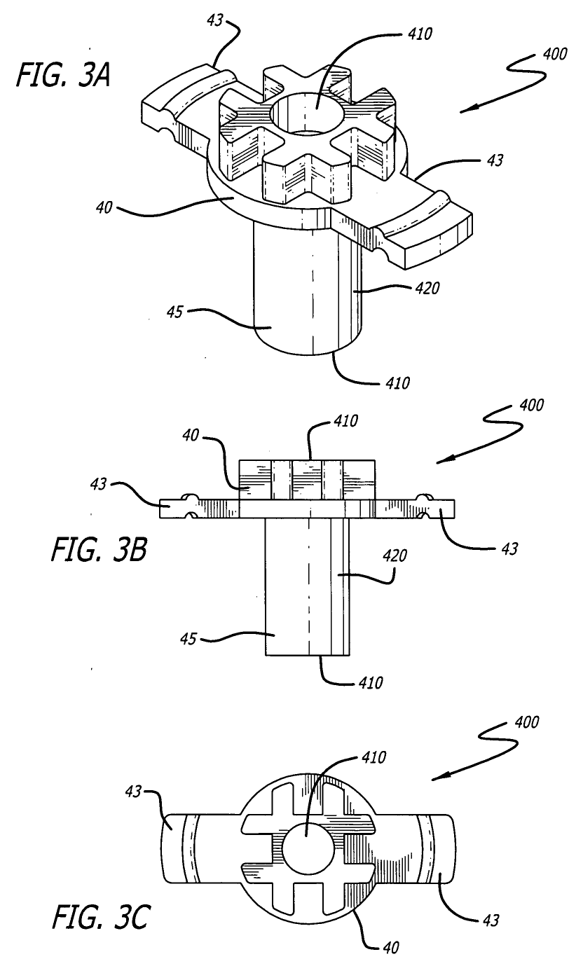

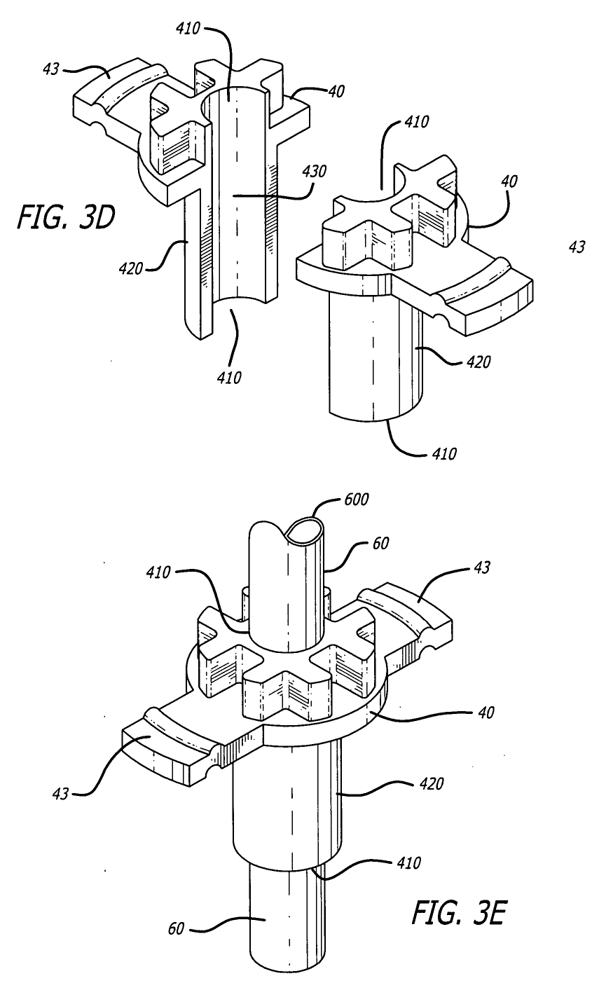

Referring now to FIGS. 3A-3E, a further aspect of the invention is a sheath plug 400. The sheath plug may include a substantially cylindrical body 420, having a substantially unobstructed longitudinal passageway 430 (FIG. 3D) having openings 410 on both ends. There may be an enlarged proximal segment 40 that may be textured for easy grasp by the clinician. The openings 410 and central passageway 430 in the sheath plug may be, in at least one embodiment, approximately twenty-two French in diameter. A twenty-two French opening 410 may snugly encircle the outer diameter of a proximal segment 60 of a respiratory assist catheter 600 (FIG. 11). A distal end 45 of the sheath plug 400 may have an outer diameter that fits snugly within the lumen or passageway of the proximal end 30 of the sheath 300 (FIG. 2). Additionally, the enlarged proximal segment 40 of the sheath plug 400 may be larger in diameter than the proximal 30 end of the sheath 300. The enlarged end 40 prevents the entire sheath plug 400 from sliding into the sheath 300. The sheath plug 400 may be made from plastics, polymers, silicone, Krayton or other elastic or pliable biocompatible materials.

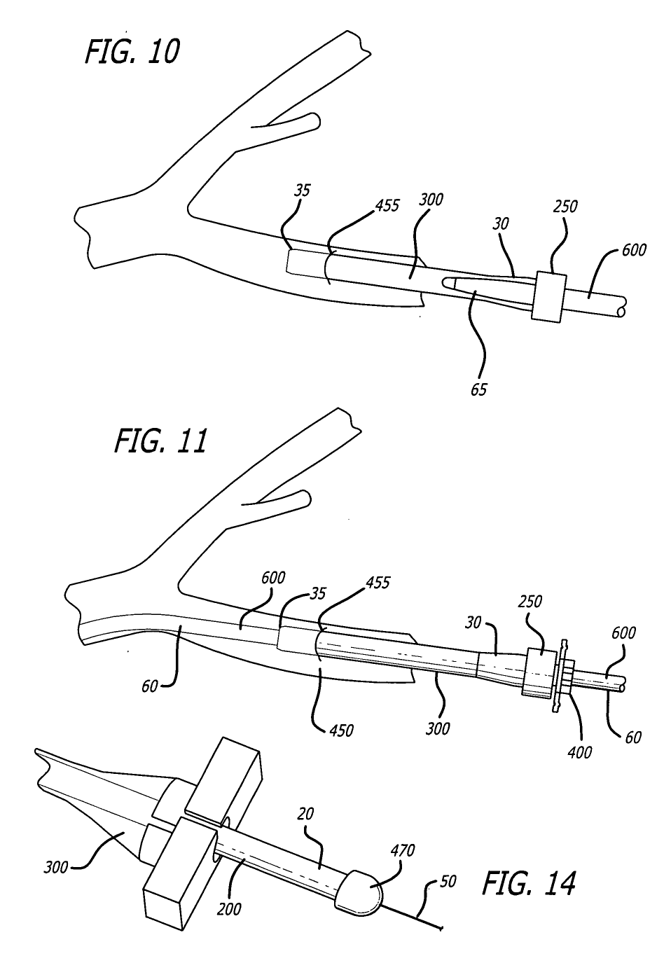

The sheath plug may be more specifically advantageously used with an intravascular medical device, which may be larger in diameter distally than proximally. An example of such a device is a respiratory assist catheter. As shown in FIGS. 10-12, the large distal end 65 of a respiratory assist catheter 600, which in one embodiment may be approximately thirty-four French in diameter, will fill most or all of the inner channel 32 of the sheath 300 and prevent back bleeding. However, the proximal segment 60 of the respiratory assist catheter 600 may be of smaller diameter than the distal segment, for example, size twenty-two French in outer diameter. Accordingly, during insertion of the respiratory assist catheter into a patient, there will be a space between the thirty-four French inner channel of the sheath 300 and the twenty-two French outer diameter of the proximal segment 60 of the respiratory assist catheter 600. Once the distal end 65 of the respiratory assist catheter 600 has been inserted into the blood vessel beyond the distal end 35 of the sheath 300, back bleeding will occur between the sheath 300 and the proximal segment 60 of the respiratory assist catheter 600. The sheath plug 400 prevents this back bleeding by filling the space between the sheath 300 and the proximal 60 respiratory assist catheter 600.

Referring to FIGS. 10 and 11, the sheath plug 400 may be preassembled over the proximal 60 respiratory assist catheter 600. Referring also to FIG. 3E, the sheath plug is configured to surround a proximal segment 60 of the respiratory assist catheter 600. The sheath plug 400 may be inserted into the sheath 300 as soon as the distal end 65 of the respiratory assist catheter 600 passes completely within the sheath 300. The sheath plug 400 should be inserted before the distal end 65 of the respiratory assist catheter 600 completely exits from the distal end 35 of the sheath 300. After the sheath plug 400 is in place, the respiratory assist catheter 600 may be passed beyond the distal 35 end of the sheath 300 into the blood vessel, without any back bleeding from the proximal 30 end of the sheath 300. The sheath plug 400 fills the space between the proximal segment 60 of the respiratory assist catheter 600 and the interior channel 32 (FIG. 2) of the proximal end 30 of the sheath 300, thereby preventing the back bleeding.

Referring more specifically now to FIG. 3D and FIG. 11, in at least one embodiment, the sheath plug 400 may be longitudinally split into at least two parts. The sheath plug 400 may be split and removed from around the proximal segment 60 of the respiratory assist catheter 600 at about the same time as the sheath 300 is split and removed from the patient. To help initiate the split, the sheath plug 400 may further include two horizontal tabs 43 at the proximal end 40. The wall of the sheath plug 400 may be axially scored (not shown), to further assist the splitting along one or more longitudinal lines of fissure. When desired by the clinician, pressure may be applied to the tabs 43, and the sheath plug 400 will split longitudinally into at least two parts. Various ways of making and using splittable medical devices are well known to those skilled in the art. Therefore, the specifics of making and using a splittable plug need not be discussed in more detail herein.

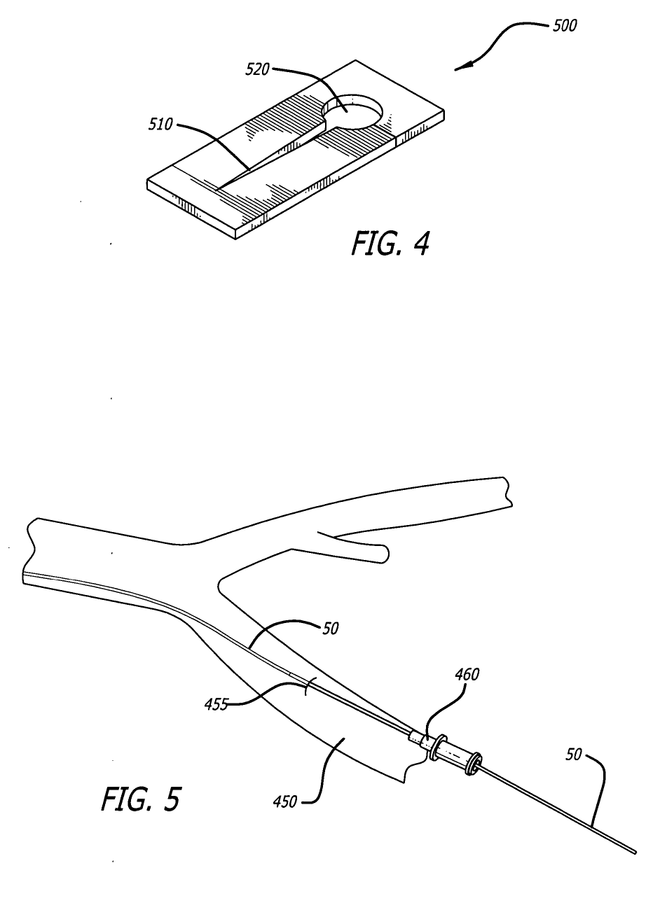

Referring now to FIG. 4 and FIG. 2, the invention further includes a slide clamp 500 for use with the sheath 300. Various clamps and clips that are well known in the art may be utilized with the sheath 300. As shown in FIG. 4, the clamp 500 may be a slide clamp. The clamp 500 is capable of closing off the inner channel 32 of the sheath 300, thereby preventing the backflow of blood through the sheath. The clamp 500 may be provided to prevent back bleeding from the sheath 300 after removal of the smaller diameter dilator 100 and the larger diameter dilator 200 from the sheath 300, but prior to insertion of an intravascular device into the sheath. The clamp 500 may be released at a time when the clinician is prepared to insert the intravascular device through the sheath 300. A small amount of back bleeding, however, may be desirable and is first permitted in order to remove air from within the sheath 300 before inserting an intravascular device.

The slide clamp 500, may be formed from, for example, plastic, metal, or other suitable materials. The slide clamp may be a thin rectangular member with a cut-out in the interior. The cut-out may be a circular hole 520 with a diameter larger than the outer diameter of the sheath 300. The cut-out may further include a slit 510, connected with and tapering away from the circular hole 520. When the sheath 300 passes through the circular hole 520, the sheath 300 is not compressed and blood flows freely through the inner channel 32 of the sheath 300. As the sheath 300 is transversely compressed into the slit 510 of the slide clamp 500, the inner channel 32 of the sheath is closed off, thereby preventing back flow of blood out of the sheath. The sheath 300 may therefore be conveniently clamped or unclamped as needed. The slide clamp may also include a notch opening (not shown) extending from the hole 520 to the outside perimeter of the clamp. This permits the sheath 300 to be inserted through the perimeter of the slide clamp 500.

Referring to FIG. 5, the invention further includes a method of dilating a percutaneous entry point 455 for the purpose of percutaneously inserting a large diameter intravenous medical device into a blood vessel. By way of example, the method described herein includes the insertion of a respiratory assist catheter 600 (FIG. 11) into a vena cava from a large peripheral vein 450, such as a femoral vein. The use of the respiratory assist catheter is by example and is not intended to be limiting. The method may be also applicable to insertion of other medical devices into the body. The insertion of the invention into a vein is also not meant to be limiting and the invention may be used for the insertion of medical devices into other body cavities or vascular structures.

To begin the method of the present invention, a large peripheral vein 450, for example, the femoral vein is identified. The femoral vein 450 may be identified next to the femoral artery (not shown). The femoral vein 450 is entered with a hypodermic needle 460 of appropriate size and length, as may be customary in medical practice. A guidewire 50 is thereafter inserted into the vein 450, through the hypodermic needle. The guidewire 50 may be advanced into the iliac vein and into the vena cava. This procedure may be performed under fluoroscopic guidance. When the guidewire 50 has been sufficiently advanced and its position confirmed, the needle is removed from the blood vessel, over the guidewire 50.

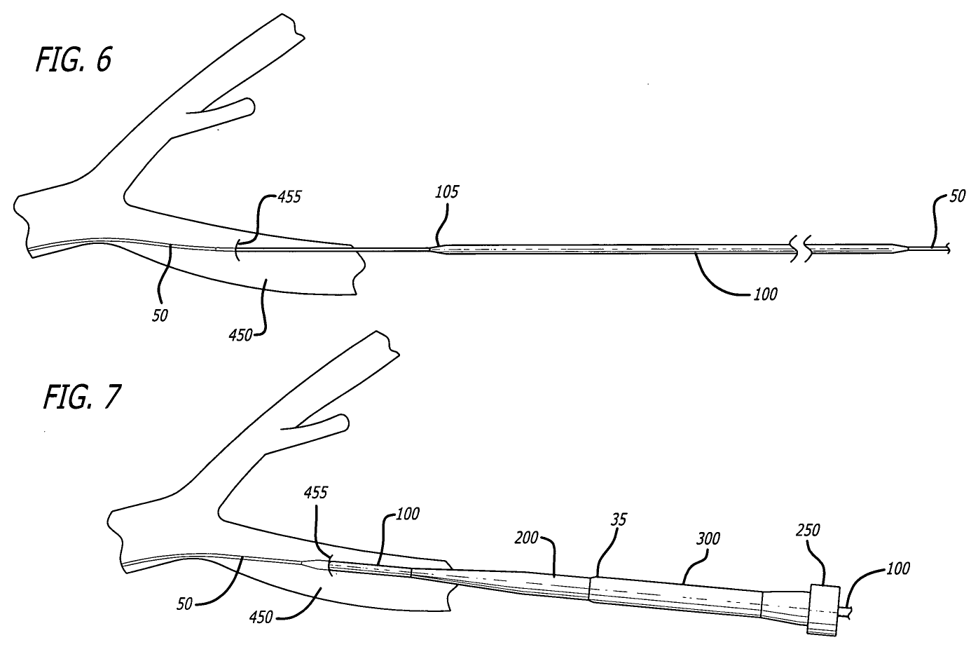

Referring to FIG. 6, the tapered distal end 105 of the smaller diameter dilator 100 is slidably advanced or passed over the guidewire 50. The smaller diameter dilator 100 is advanced over the guidewire 50 through a percutaneous entry point 455 and into the vein 450. The smaller diameter dilator 100 is typically advanced to approximately the midpoint of the total length of the smaller diameter dilator 100. In at least one embodiment, this still leaves approximately eighteen inches (forty-six cm) of a thirty-six inch (ninety-two cm) long smaller diameter dilator 100 extending out of the percutaneous entry point 455. This length is more than sufficient for the clinician to retain control of the smaller diameter dilator 100.

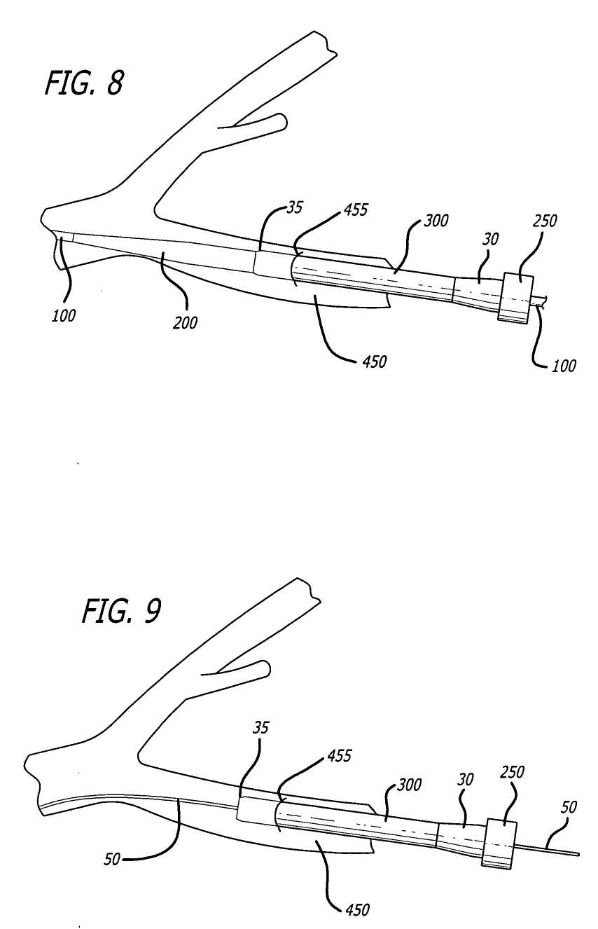

Referring to FIGS. 7 and 8, the pre-assembled and detachably locked together larger diameter dilator 200 and sheath 300 are then slidably advanced over the guidewire 50 and the smaller diameter dilator 100 up to the percutaneous entry point 455. The larger diameter dilator 200 and sheath 300 assembly continue to be inserted over the smaller diameter dilator 100 into the patient until the sheath 300 has entered into the vein 450. The sheath 300 is typically advanced to approximately the iliac vein. In one embodiment, the smaller diameter dilator 100, larger diameter dilator 200, and sheath 300 are assembled together before insertion of the smaller diameter dilator 100 into the vein 450.

Referring now to FIG. 9, after insertion of the sheath 300 into the vein 450, the sheath 300 is unlocked from the larger diameter dilator 200. The guidewire 50, smaller diameter dilator 100, and larger diameter dilator 200 are then removed, preferably together and maintaining approximately the same relative position to each other during their removal. At the same time, the clinician secures the sheath 300 in position in the patient. As the guidewire 50, smaller diameter dilator 100 and larger diameter dilator 200 are removed from the sheath 300, a clamp may be applied to the sheath 300. When using a slide clamp 500 (FIG. 4), the sheath 300 is force into the slit 510 of the clamp 500, thereby closing off a portion of the inner channel 32 (FIG. 2) of the sheath 300. A small amount of back bleeding from the sheath 300 may be acceptable and desirable to allow any trapped air to escape from the sheath 300, thereby avoiding an air embolism in the patient.

Referring now to FIGS. 10-12, the clamp 500 (FIG.4) remains in place, compressing the sheath 300, until the clinician is ready to insert the respiratory assist catheter 600. The distal end 65 of the respiratory assist catheter 600 may now be inserted into the proximal end 30 of the sheath 300. The slide clamp 500 (FIG. 4) may be released by moving the sheath 300 from the slit 510 into the circular hole 520 to allow the respiratory assist catheter 600 to slide distally into the sheath 300. As the respiratory assist catheter 600 passes into the distal portion 35 of the sheath 300, the sheath plug 400 may be connected into the proximal 30 end of the sheath 300. The respiratory assist catheter 600 may now be further inserted beyond the distal end 35 of the sheath 300 into the desired position in the vena cava. When satisfactory positioning of the respiratory assist catheter 600 is verified, the sheath 300 may be split and removed, and the sheath plug 400 may be also split and removed. Only the respiratory assist catheter 600 remains indwelling in the patient. The respiratory assist catheter 600 may now be secured in place, for example, by suturing it to the skin.

Referring now to FIG. 13, yet another aspect of the invention is a kit including a guidewire 50, at least two dilators (for example, a first smaller diameter dilator 100 and a second larger diameter dilator 200), a sheath 300, and a sheath plug 400.

Referring now to FIG. 14, yet another aspect of the present invention is a hemostasis seal 470 for use with larger dilators, for example, the second larger diameter dilator 200. The larger diameter dilator 200 may be used without the smaller diameter dilator I 00 (FIG. 1). The proximal end 20 of the dilator is configured with the hemostasis seal 470. The diameter of the dilator 200 inner channel 22 (FIG. 2) is substantially larger than the outer diameter of the main body of the guidewire 50. If the larger diameter dilator 200 is used without the smaller diameter dilator 100 (FIG. 2), there is a large space between the guidewire 50 and larger diameter dilator 200 for leakage of blood. Thus, the hemostasis seal 470 is designed to prevent blood leakage from the dilator 200 while the guidewire 50 protrudes from the proximal end 20 of the larger diameter dilator 200. The seal 470 may be formed as a cap disposed over or within the proximal end 20 of the dilator 200. The cap or seal 470 may be formed from a natural or synthetic rubber, such as silicone, or other suitable material that will form a fluidic seal (blood tight) around the guidewire. The cap or seal 470 may be slit or punctured so as to ease insertion of the proximal end of the guidewire 50 through the dilator 200 and hemostasis seal 470.

Once the guidewire 50 is properly positioned, the introducer assembly may be inserted into the patient's vasculature by threading the dilator 200 along the guidewire 50 until the distal end 25 and distal aperture 220 (FIG. 2) of the dilator 200 reaches the desired location in the vasculature. As shown in FIG. 14, the proximal portion of the guidewire 50 extends through the proximal end 20 of the dilator 200 and hemostasis seal 470. The dilator 200 may be removed from the vasculature prior to attachment of a medical device to the proximal end of the guidewire 50 or prior to a medical device being inserted through the sheath 300 and deployed over the guidewire 50 and into the patient's vasculature.

While a particular forms of the present invention have been illustrated and described, it will also be apparent to those skilled in the art that various modifications can be made without departing from the inventive concept. The described embodiments are to be considered in all respects only as illustrative and not restrictive. References to use of the invention with the respiratory assist catheter are by way of example, and the invention may be used for insertion of other medical devices into veins, arteries, and the body in general. Other materials of construction and dimensions of the dilator assembly and introducer assembly may be used. The present invention may be embodied in other specific forms without departing from its spirit or essential characteristics. Accordingly, it is not intended that the invention be limited except by the appended claims.

We claim:

1. A dilator assembly, comprising:
a first dilator configured with a first proximal portion having a first proximal aperture, a first tapered distal portion having a first distal aperture, and a first tubular segment having a substantially uniform first outer diameter and a first inner lumen between the first proximal portion and the first distal portion; and
a second dilator configured with a second proximal portion having a second proximal aperture, a second tapered distal portion having a second distal aperture, and a second tubular segment between the second proximal portion and the second distal portion, the second tubular segment having a substantially uniform second outer diameter and a second inner lumen, wherein the diameter of the second inner lumen is substantially the same as the outer diameter of the first tubular segment of the first dilator.

2. The dilator assembly of claim 1, at least one successive dilator configured with a proximal portion having a proximal aperture, a tapered distal portion having a distal aperture, and a tubular segment between the proximal portion and the distal portion, the tubular segment having a substantially uniform outer diameter and a inner lumen, wherein the diameter of each inner lumen of each successive dilator is configured with a diameter substantially the same as an outer diameter of another preceding dilator of the assembly.

3. The dilator assembly of claim 1, further including a hemostasis seal configured to be disposed in at least one of the first proximal aperture of the first dilator and the second proximal aperture of the second dilator.

4. An introducer assembly, comprising:
a first dilator configured with a first proximal portion having a first proximal aperture, a first tapered distal portion having a first distal aperture, and a first tubular segment having a substantially uniform first outer diameter and a first inner lumen between the first proximal portion and the first distal portion;
a second dilator configured with a second proximal portion having a second proximal aperture, a second tapered distal portion having a second distal aperture, and a second tubular segment between the second proximal portion and the second distal portion, the second tubular segment having a substantially uniform second outer diameter and a second inner lumen, wherein the diameter of the second inner lumen is substantially the same as the outer diameter of the first tubular segment of the first dilator; and
a sheath configured with a third inner diameter substantially the same as the second outer diameter of the second dilator.

5. The introducer assembly of claim 4, further including a sheath plug having a substantially cylindrical body with a distal portion having an outer diameter substantially the same as the third inner diameter of the sheath and an enlarged proximal segment.

6. The introducer assembly of claim 5, wherein the sheath plug further includes at least two tabs disposed on the enlarged proximal segment, and wherein the cylindrical body is configured to tear along a longitudinal axis.

7. The introducer assembly of claim 4, further including a fitting configured to detachably lock the proximal end of the sheath to the proximal end of the second dilator.

8. The introducer assembly of claim 4, further including a slide clamp configured to close a lumen in the sheath.

9. A method of dilating an opening into a blood vessel, comprising:
inserting a guidewire into a blood vessel;
inserting a first dilator into blood vessel over the guidewire; and
inserting a second dilator into blood vessel over the first dilator.

10. The method of claim 9, further including inserting at least one additional dilator into blood vessel over each other dilator.

11. A method of inserting a medical device into a blood vessel, comprising:
inserting a guidewire into a blood vessel;
sliding a first dilator over the guidewire;
sliding a second dilator over the first dilator;
inserting a sheath over the second dilator;
removing the first dilator and the second dilator from the blood vessel; and
inserting a medical device into the blood vessel through a proximal opening of the sheath.

12. The method of claim 11, further comprising splitting the sheath so as to remove the sheath from the blood vessel.

13. The method of claim 12, further comprising:
inserting a sheath plug over a proximal segment of the medical device so as to from a substantially blood tight seal at the proximal opening of the sheath when the medical device is inserted into the blood vessel; and
splitting the sheath plug into so as to remove the sheath plug from the medical device.

14. A method of inserting a medical device into a blood vessel, comprising:
inserting a guidewire into a blood vessel;
sliding a first dilator over the guidewire;
sliding a second dilator over the first dilator;
inserting a medical device (cannula, catheter, or other) over the second dilator and into the blood vessel
removing the first dilator and the second dilator from the blood vessel.

15. A dilator kit, comprising:
a guidewire;
a first dilator configured with a first proximal portion having a first proximal aperture, a first tapered distal portion having a first distal aperture, and a first tubular segment having a substantially uniform first outer diameter and a first inner lumen between the first proximal portion and the first distal portion; and
a second dilator configured with a second proximal portion having a second proximal aperture, a second tapered distal portion having a second distal aperture, and a second tubular segment between the second proximal portion and the second distal portion, the second tubular segment having a substantially uniform second outer diameter and a second inner lumen, wherein the diameter of the second inner lumen is substantially the same as the outer diameter of the first tubular segment of the first dilator.

16. The dilator kit of claim 15, further including a sheath configured with a third inner diameter substantially the same as the second outer diameter of the second dilator.

17. The dilator kit of claim 16, further including a sheath plug configured to form a substantially fluid tight seal in a proximal opening of the sheath.
